(19) United States
(12) Patent Application Publication
(10) Pub. No.: US 2025/0169804 A1
(43) Pub. Date: May 29, 2025

(54) MEDICAL DEVICES, COMPONENTS, AND METHODS FOR REUSABLE DEVICES OR COMPONENTS

(71) Applicants: BOSTON SCIENTIFIC MEDICAL DEVICE LIMITED, Galway (IE); Boston Scientific Scimed, Inc., Maple Grove, MN (US)

(72) Inventors: Pooja Bhuvanesh KULKARNI, Pune (IN); Barry WEITZNER, Acton, MA (US); Deepak Kumar SHARMA, Muzaffarnagar (IN); Shrikant Vasant RAUT, Mumbai (IN); Aditya DHANOTIYA, Indore (IN); Nachiket GOLE, Pune (IN); Subodh MOREY, Ponda (IN)

(73) Assignees: BOSTON SCIENTIFIC MEDICAL DEVICE LIMITED, Galway (IE); Boston Scientific Scimed, Inc., Maple Grove, MN (US)

(21) Appl. No.: 18/962,033

(22) Filed: Nov. 27, 2024

Related U.S. Application Data
(60) Provisional application No. 63/603,775, filed on Nov. 29, 2023.

(51) Int. Cl. A61B 10/06; A61B 17/00; A61B 17/12; A61B 17/128

(52) U.S. Cl. CPC A61B 10/06; A61B 17/1285; A61B 2017/0023; A61B 2017/00469; A61B 2017/00473; A61B 2017/12004

(57) ABSTRACT

A medical device is disclosed herein. The medical device including a disposable portion. The disposable portion including a sheath and a wire disposed within a lumen of the sheath. The medical device further including a handle. The handle including a main body with a slot and a releasably engageable moveable body. The moveable body is formed by a first component and a second component. The first component and the second component are releasably engageable with both the slot and a proximal portion of the disposable portion. Movement of the moveable body is configured to move the wire.

### CROSS-REFERENCE TO RELATED APPLICATIONS

This application claims the benefit of priority to U.S. Provisional Application No. 63/603,775, filed on Nov. 29, 2023, which is incorporated by reference herein in its entirety.

### TECHNICAL FIELD

Various aspects of this disclosure relate generally to medical devices, components, and methods for reusable devices or components. More specifically, aspects of the disclosure pertain to devices, components, and methods for releasably coupling devices or components to form a medical device.

### BACKGROUND

Single-use medical devices are disposed of after use, which can result in a large amount of waste and create a burden on the environment. Many endoscopic devices, including, for example, hemostasis clips and biopsy forceps, are currently single-use devices. Such devices often include a handle, a shaft, and a distal tip that includes an end effector. The distal tip and the shaft are inserted in the body to perform a procedure at a target site (e.g., to obtain a tissue sample), while the handle remains outside of the body. Disposal of the entire device creates waste, imposing an environmental burden. Therefore, there is a need for devices or components that result in less waste and/or environmental burden. The devices, components, and methods described herein may alleviate this deficiency and one or more other deficiencies in the art. However, the attached claims specify the scope of this disclosure rather than the ability to solve any specific problem.

### SUMMARY

Each of the aspects disclosed herein may include one or more of the features described in connection with any of the other disclosed aspects.

In one or more aspects, the disclosure includes medical devices and related methods useful for endoscopic procedures. For example, the disclosure includes a medical device that may include a disposable portion, which may include a sheath and a wire disposed within a lumen of the sheath. The medical device also may include a handle. The handle may include a main body with a slot. The medical device may further include a releasably engageable moveable body. The moveable body may be formed by a first component and a second component. The first component and the second component may be releasably engageable with both the slot and a proximal portion of the disposable portion. Movement of the moveable body may be configured to move the wire.

The medical device may include one or more of the following aspects. The disposable portion may include an end effector. The moveable body may be configured to receive a proximal portion of the wire and movement of the moveably body may be configured to actuate the end effector. The disposable portion may include a first crimp positioned at a proximal end of the wire. The moveable body may be configured to receive the first crimp. The first component may include a release tab. When the release tab is in an actuated configuration, the first crimp and proximal end of the wire may be receivable within a recess of the second component. The release tab may include a tab. A slot may be defined through the tab. The slot may be sized and shaped to receive the wire and reject the first crimp. When the first crimp is received within the recess and the release tab is in a deactuated configuration, the first crimp may be prevented from moving in a distal direction by the tab. When the first crimp is received within the recess and the release tab is in a deactuated configuration, a portion of the wire may be disposed within the slot of the tab. The handle may include a locking mechanism configured to couple the disposable portion with the handle.

The disposable portion may include a removable distal tip. The distal tip may include a lumen and a chamber. The lumen and chamber may be in communication with each other and may be positioned along a central longitudinal axis of medical device. At least a portion of the wire may be positioned within the lumen and the chamber. The disposable portion may include a second crimp positioned distally of the first crimp on the wire. The second crimp may be fixedly attached to a portion of the sheath and may be positioned within the chamber. A dimension of the second crimp may prevent the second crimp from exiting the chamber. The second crimp may prevent movement of the sheath relative to the distal tip.

A distal tip of the disposable portion may include a protrusion extending in a proximal direction from a proximal end of the distal tip. The main body may include a chamber configured to receive the protrusion. The distal tip may be releasably attached to the main body when the protrusion is received within the chamber of the main body. The protrusion may include a flange and the chamber may include a flange. The protrusion of the distal tip may be received within the chamber of the main body. The flange of the protrusion and the flange of the chamber may contact one another to prevent the protrusion from exiting the chamber. The flange of the protrusion may extend radially outward relative to a central longitudinal axis of the medical device and the flange of the chamber may extend radially inward relative to the central longitudinal axis of the medical device.

In one or more aspects, the disclosure also includes a medical device that may include a disposable portion, and the disposable portion may include a distal tip. The distal tip may include a cavity at a proximal end of the distal tip. The medical device also may include a handle. The handle may include a main body with a slot. The main body may include a protrusion extending distally from a distal end of the main body. The cavity may be configured to receive and contain the protrusion of the distal tip when the disposable portion is in an engaged configuration with the main body. The medical device may further include a moveable body. Movement of the moveable body may be configured to control an end effector of the medical device.

The medical device may include one or more of the following aspects. The protrusion may include a tab extending radially outward relative to a central longitudinal axis of the medical device. The cavity may include a tab extending radially inward relative to the central longitudinal axis of the medical device. The disposable portion may include a sheath. The sheath may be receivable within a channel of the moveable body. The sheath may include a flange and the channel including a flange. The flange of the moveable body may be positioned distally to the flange of the sheath when sheath is received within the moveable body. The moveable body may be formed from a first moveable body component and a second moveable body component. The moveable body may be releasably engageable to the main body and the slot.

In one or more another aspects, the disclosure further includes a method of releasably engaging components of medical device. The medical device may include a reusable portion including a handle with an actuator. The actuator may be a moveable body. The medical device may include a disposable portion. The disposable portion may include a wire and a crimp positioned at a proximal end of the wire. The moveable body may include a release tab and a recess configured to receive the crimp. A tab of the release tab may be distal to the recess and blocks insertion of the crimp into the recess when the release tab is in a deactuated state. In an actuated state, the tab of the release tab may no longer block insertion of the crimp into the recess. The method may include one or more of coupling the disposable portion to the reusable portion; inserting components of the disposable portion into a bodily lumen of a patient; treating a treatment site; removing components of the disposable portion from the bodily lumen of the patient; and uncoupling the disposable portion from the reusable portion.

### BRIEF DESCRIPTION OF THE FIGURES

The accompanying drawings, which are incorporated in and constitute a part of this specification, illustrate various aspects of this disclosure and together with the description, serve to explain the principles of the disclosure.

FIG. 1 depicts an exemplary medical device.

FIG. 2 depicts a cross sectional view of a proximal portion of the medical device shown in FIG. 1.

FIG. 3 depicts a proximal to distal view of the medical device of FIG. 1.

FIG. 4 depicts another exemplary medical device.

FIG. 5A depicts yet another exemplary medical device.

FIG. 5B depicts an internal locking mechanism of the exemplary medical device shown in FIG. 5A.

FIG. 6 depicts a further exemplary medical device.

FIG. 7 depicts yet a further exemplary medical device.

FIG. 8 depicts an exemplary method flowchart.

### DETAILED DESCRIPTION

Reference is now made in detail to examples of this disclosure, aspects of which are illustrated in the accompanying drawings. Wherever possible, the same reference numbers will be used throughout the drawings to refer to the same or like parts.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. Several drawings include arrows labeled “P” and “D,” indicating proximal and distal directions, respectively.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

The embodiments, and components thereof, shown in the figures of this disclosure may not be drawn to scale.

Embodiments of this disclosure include a medical device that has a first component that may be releasably coupled to a second component. The first component may include a disposable portion including a distal tip and, a wire (e.g., an actuation or pull wire), and an end effector. The disposable portion may be placed in sterile packaging until use. Distal tip may be releasably engaged with the wire. The second component may include a handle including a main body and a releasably engageable spool (e.g., an actuator). Prior to use of the medical device, the wire of the first component may releasably attach to the spool of the second component, and the distal tip may releasably attach to the main body. After use, the first component may be uncoupled from the second component. For example, the wire of the first component may be uncoupled from the spool, and the distal tip may be uncoupled from the main body. The distal tip may be uncoupled from the wire. In this way, the first component, components of which contact the patient during use, may be disposed of. Contrarily, the second component may be cleaned as needed and/or re-used with another first component in a subsequent procedure.

Such aspects of medical devices, components, and methods may help to reduce waste and/or reduce overall cost of the medical device (fewer handles needed for a number of procedures, less packaging needed for additional handles, etc.). Such aspects also may result in a universal proximal component (including a handle) that may be used with numerous different types of distal components (including different types of end effectors). The handle (e.g., main body and spool) may be operated outside the body of the patient in a sterile field, while the insertion portion (e.g., wire, sheath, and end effector) may be inserted into and operated within the body of the patient. Further, insertion portion may be inserted into a port (such as an accessory port) of a scope. The insertion portion may be disposed of and replaced with a new insertion portion for another treatment or procedure, for example, to help prevent infection, cross-contamination, and the like.

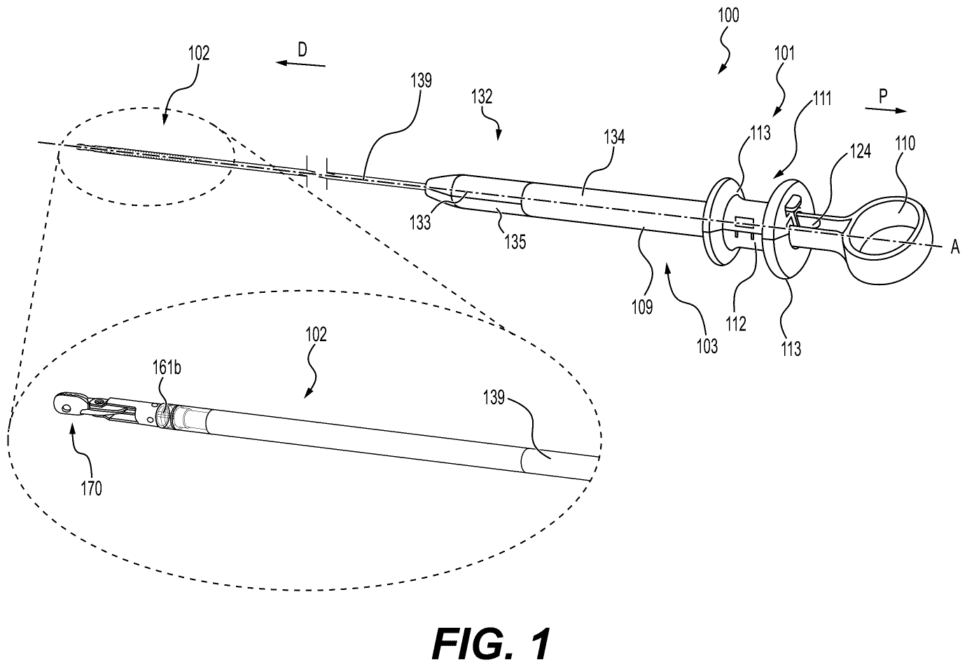

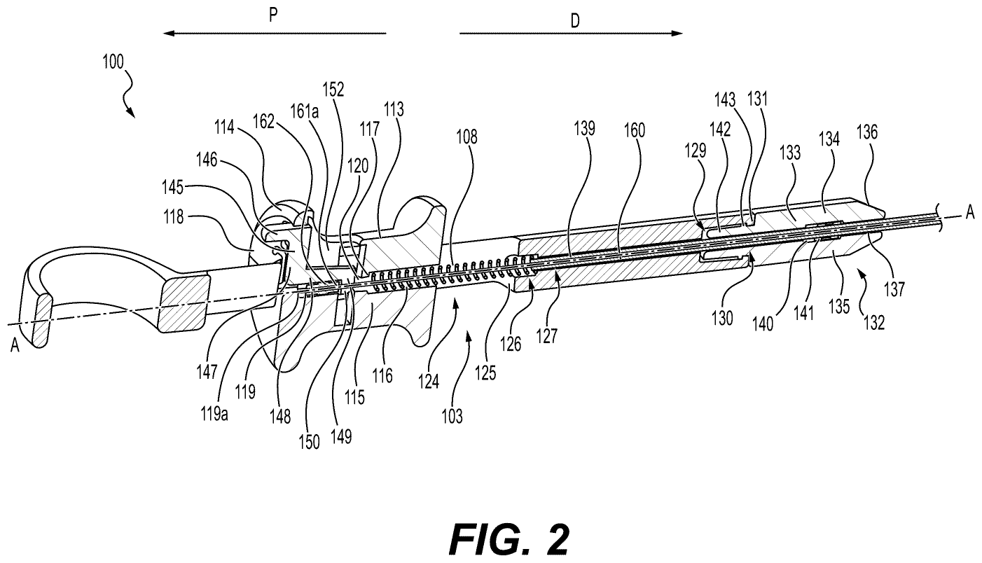

FIG. 1 illustrates a medical device 100, including a proximal portion 101 and a distal portion 102. FIG. 1 includes an enlarged view of distal portion 102. As shown in FIG. 1, proximal portion 101 includes a handle 103. Medical device 100 includes a disposable portion 132, for example, extending from a distal end of handle 103. Medical device 100 (e.g., a portion of disposable portion 132) may be delivered to a treatment site via an insertion device (e.g., through a working channel of an endoscope, ureteroscope, catheter, etc.). Disposable portion 132 may be releasably coupled to handle 103. Disposable portion 132 may include or otherwise radially surround a wire 160 (FIG. 2), for example, a pull wire or an actuation wire. For example, with disposable portion 132 coupled to handle 103, wire 160 may extend from handle 103 through disposable portion 132. Wire 160 may be moveable (e.g., within handle 103 and disposable portion 132) via manipulation of one or more portions of handle 103. Medical device 100 also includes end effector 170 at a distal end of disposable portion 132, for example, at a distal end of distal portion 102. For example, end effector 170 may be coupled to (e.g., directly or indirectly) to a distal end of wire 160 (as seen in FIG. 2). In these aspects, manipulation of one or more portions of handle 103 may control one or more aspects of distal portion 102 of medical device 100, for example, control one or more aspects of end effector 170.

Handle 103 includes a main body 109, for example, including a ring 110 (e.g., a thumb ring), for example, at a proximal end of main body 109. Handle 103 also includes a first moveable member/body or spool 111. Spool 111 may be an actuator and may include an indented portion 112 (e.g., with a relatively smaller lateral cross-section) and one or more (e.g., two) ridged or extended portions 113 (e.g., with a relatively larger lateral cross-section). In these aspects, indented portion 112 may receive one or more of the user's fingers, such that movement of the user's finger(s) controls the movement of spool 111. Main body 109 may include a slot 124, for example, extending longitudinally through a portion of main body 109, for example, from a position spaced distally from ring 110. A portion of spool 111 may extend into a portion of slot 124, such that spool 111 is moveable (e.g., longitudinally moveable distally and/or proximally) along slot 124. In these aspects, slot 124 may define a range of movement for spool 111. As discussed in detail below, spool 111 may be moveable within slot 124, for example, proximally and/or distally, to control one or more aspects of end effector 170. Furthermore, as shown in FIG. 2, slot 124 may include a wide portion 125, a tapered or narrow lumen 126, and more tapered or narrower lumen 127. Narrower lumen 127 may include a smaller diameter than narrow lumen 126. As discussed below, wide portion 125 may be proximal of narrow lumen 126, and narrow lumen 126 may be proximal of narrower lumen 127. It should be understood that spool 111, any other spools discussed below, may be formed in a variety of sizes to accommodate users with different sized hands or fingers and/or to accommodate different needs of different users.

FIG. 2 illustrates a longitudinal cross-sectional view of various portions of medical device 100, including handle 103. A distal end of main body 109 may include a chamber 129. Chamber 129 may be connected to a distal end of narrower lumen 127 of slot 124. Chamber 129 may include a distal opening 130. Chamber 129 may include a flange 131, for example, protruding radially inward, at a distal end of chamber 129. Flange 131 may help to narrow or reduce the cross-sectional size of distal opening 130. Chamber 129 and slot 124 (e.g., wide portion 125, narrow lumen 126, and narrower lumen 127) may be configured to receive a proximal portion of the disposable portion 132.

Disposable portion 132 may include a distal tip 133. Distal tip 133 may be a removable distal tip. Distal tip 133 may include a proximally extending extension 142 that extends from a proximal end of distal tip 133. Extension 142 may cover and/or surround a portion of sheath 139. Extension 142 may be received within chamber 129 and releasably engage with chamber 129. Extension 142 may include a tab 143 that extends radially outward relative to a central longitudinal axis (labeled as axis A in FIGS. 1-3) of medical device 100. Tab 143 may be positioned distally of flange 131 when extension 142 is positioned within chamber 129. Tab 143 and flange 131 may contact one another and facilitate the releasable engagement of main body 109 and distal tip 133. Main body 109 and distal tip 133 may be engaged by pushing extension 142 into chamber 129 until tab 143 is distal to flange 131. Main body 109 and distal tip 133 may be disengaged or uncoupled by manually pulling apart main body 109 and distal tip 133, e.g., by urging main body 109 proximally and/or urging distal tip 133 distally. Distal tip 133 may be rotatable relative to main body 109. Main body 109 may include a recess 107 at a distal end of main body 109. Recess 107 may expose a portion of the distal facing surface of distal tip 133. Recess 107 may be sized and/or shaped to receive a portion of a digit (e.g. a finger) of a user therein to help remove distal tip 133. A force may be applied against the exposed portion of the distal facing surface of distal tip 133 to decouple distal tip 133 from main body 109.

As mentioned, medical device 100 includes disposable portion 132. Disposable portion 132 may include the distal tip 133. Distal tip 133 may be comprised of a first distal tip component 134 and a second distal tip component 135. A distalmost portion 136 of distal tip 133 may taper or decrease in diameter in the proximal to distal direction. Distal tip 133 may include a lumen 137 extending along axis A. Although distal tip 133 is discussed through this disclosure as being disposable, in some embodiments, distal tip 133 may be cleaned/decontaminated and/or otherwise reused similar to handle 103 (e.g., main body 109 and spool 111).

Disposable portion 132 may include sheath 139. Sheath 139 includes a lumen configured to receive wire 160, for example, an actuation or pull wire for actuation of end effector 170. At least a portion of sheath 139 and wire 160 may be disposed within lumen 137 of distal tip 133 and slot 124. Wire 160 may include a proximal end 161a (FIG. 2) and a distal end 161b (FIG. 1). Proximal end 161a may be coupled to spool 111. Proximal end 161a may include a crimp 162. Crimp 162 may be fixedly or removably attached to proximal end 161a. Crimp 162 may have a larger diameter than wire 160.

Distal tip 133 may include an inner chamber 140 in communication with lumen 137, and inner chamber 140 may be disposed along axis A. Distal tip 133 may include a crimp 141 disposed within inner chamber 140. Crimp 141 may be configured to contact and/frictionally engage sheath 139. Crimp 141 may be stationary relative to distal tip 133 to prevent sheath 139 from moving relative to crimp 141 and/or relative to distal tip 133.

Spool 111 may be removably, or releasably, engageable with slot 124. Spool 111 may be formed by a first spool component 114 (e.g., a first moveable body component) and a second spool component 115 (e.g., a second moveable body component). Spool 111 may be approximately divided along a plane including axis A of medical device 100, for example, to form first spool component 114 and second spool component 115. Spool 111 may include a cavity 116. Respective portions of each of first spool component 114 and second spool component 115 may form cavity 116. Cavity 116 may be sized and/or shaped to receive at least a portion of biasing element 108 and at least a portion of wire 160. Cavity 116 may include a distal facing surface 117 at a proximal end of cavity 116. A proximal end of wire 160 may contact and/or extend proximally of distal facing surface 117.

Medical device 100 may include a biasing element 108, for example, as a part of disposable portion 132 or as a part of handle 103 (e.g. spool 111 and/or main body 109). The transition between narrow lumen 126 and narrower lumen 127 may help support or otherwise provide a stop surface for a distal end of biasing element 108. Biasing element 108 may be a spring. Biasing element 108 may be a sized to have a diameter slightly smaller than a diameter of narrow lumen 126, for example, so that narrow lumen 126 may receive biasing element 108. The distal facing surface 117 may provide a stop surface for a proximal end of biasing element 108.

First spool component 114 may include a release tab 145. Release tab 145 may be a separate component or may be an internal portion of (e.g., integrally formed with) first spool component 114. Release tab 145 may include a lever portion 146 extending proximally beyond a proximal facing surface 118 of spool 111. A proximal portion of lever portion 146 may form a portion of the proximal facing surface 118 of spool 111. Release tab 145 may include a stem portion 147. Stem portion 147 may extend from a distal end/distal portion of lever portion 146. Lever portion 146 may be connected to stem portion 147 at a radially outer end (relative to axis A) of stem portion 147. Stem portion 147 may extend radially inward toward axis A of medical device 100. Stem portion 147 and lever portion 146 be approximately perpendicular to one another, forming an approximately right angle between stem portion 147 and lever portion 146. A proximal surface of stem portion 147 may form a portion of proximal facing surface 118. Release tab 145 may include an engaging portion 148. Engaging portion 148 may extend distally of an radially inner (relative to axis A) end of stem portion 147 opposite to the radially outer end of the stem portion 147 connected to lever portion 146. Engaging portion 148 may extend distally and/or at an angle toward axis A of medical device 100. A top surface of engaging portion 148 may extend distally at an angle (e.g., an acute angle) toward axis A of medical device 100, and a bottom surface of engaging portion 148 may extend distally, parallel with axis A. In some embodiments, the top surface of engaging portion 148 may extend distally, parallel with axis A and the bottom surface of engaging portion 148 may extend distally at an angle toward axis A.

Engaging portion 148 may include a tab 149. Tab 149 may extend from a distal end of engaging portion 148. Tab 149 may extend radially inward toward axis A of medical device 100. Tab 149 may include a notch 150. Notch 150 may extend along axis A of the medical device 100. Notch 150 is sized and shaped to receive wire 160 but not receive (e.g., reject) crimp 162. In other words, notch 150 may be narrower than a diameter/major dimension of crimp 162, so that crimp 162 may not travel through (e.g., move longitudinally through) notch 150. Second spool component 115 may include a recess 119. Recess 119 may be sized and shaped to receive crimp 162. Recess 119 may include a distal facing surface at a proximal end of recess 119. When the first spool component 114 and second spool component 115 are in an engaged configuration (as at least shown in FIG. 1) crimp 162 may be retained within recess 119 proximal of tab 149. In the engaged configuration, when spool 111 is urged in a distal direction, crimp 162 may contact and/or be urged distally by the distal facing surface 119a at the proximal end of recess 119. When spool 111 is urged in a proximal direction, crimp 162 may contact and be urged by tab 149. Spool 111 may include a lumen 120 positioned between recess 119 and cavity 116. Lumen 120 may be configured to receive the crimp 162 therethrough.

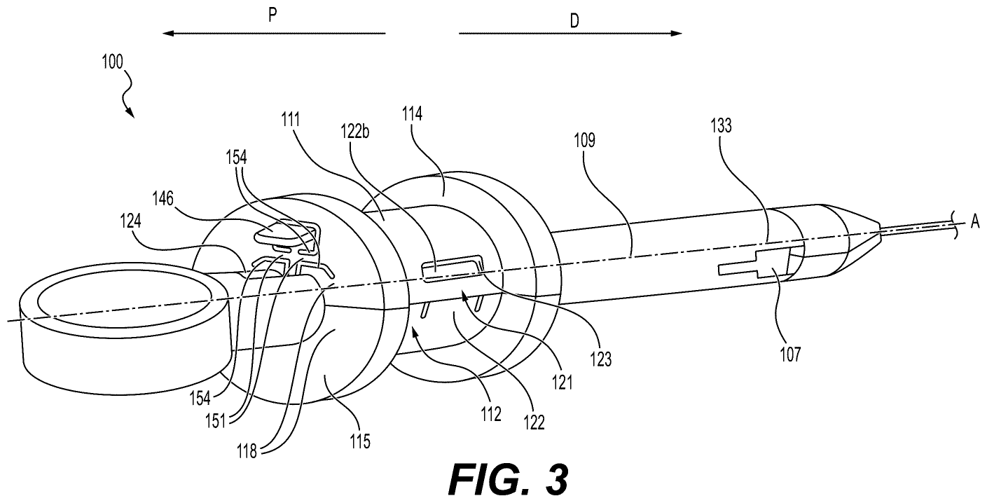

FIG. 3 depicts a longitudinal view of various portions of medical device 100. The proximal facing surface 118 of spool 111 may include one or more apertures 154 defined therethrough in a distal direction. One of the one or more apertures 154 may extend along a portion of the periphery of the lever portion 146. Another one of the one or more apertures 154 may extend along a portion of the periphery of the stem portion 147. One or more apertures 154 may be arranged to define a pivot bar 151. Pivot bar 151 may be attached to stem portion 147 so that release tab 145 may pivot about pivot bar 151 when a force in the direction of axis A of the medical device 100 is applied on lever portion 146. One or more apertures 154 may facilitate rotation of release tab 145 about pivot bar 151. While pivot bar 151 is described and shown as being integrally formed with proximal facing surface 118, it should be understood that pivot bar 151 may be a separate component. Spool 111 may include a cavity 152 (as seen in FIG. 2). A portion of release tab 145 may be disposed within cavity 152. The dimensions of cavity 152 may limit and/or facilitate the rotation of release tab 145 about pivot bar 151.

First spool component 114 may be releasably attached to second spool component 115 via a snap 121, for example, in positioned on indented portion 112. Snap 121 may include a cantilevered portion 122 that includes a protrusion 122b. Snap 121 may also include a receiving slot 123 for receive a portion of protrusion 122b of cantilevered portion 122. First spool component 114 may include slot 123, and second spool component 115 may include cantilevered portion 122. In the alternative, first spool component 114 may include cantilevered portion 122, and second spool component 115 may include slot 123. It should be understood that snap 121 and its corresponding components may be any releasable snap known by those skilled in the art. Snap 121 may be engaged when the first spool component 114 and second spool component 115 is in the engaged configuration. Snap 121 may be disengaged by depressing protrusion 122b and pulling first spool component 114 and second spool component 115 apart.

First spool component 114 and second spool component 115 may be releasably engageable with main body 109 (e.g. with slot 124) and the proximal portion of disposable portion 132 (e.g. a proximal portion of the wire 160 and crimp 162). First spool component 114 and second spool component 115 may be transitioned to an engaged configuration by aligning components 114, 115 about main body 109 so that a portion of each component 114, 115 is positioned within slot 124 and at least one portion of each component 114, 115 is positioned outside slot 124 and around main body 109. After aligning components 114, 115, components 114, 115 may be releasably engaged with one another about slot 124. Snap 121 may be engaged, preventing spool 111 from detaching from main body 109. Spool 111 may be releasably engaged proximal to the crimp 162 of the disposable portion 132. Spool 111 may be advanced distally along slot 124, so that crimp 162, biasing element 108, and/or wire 160 enter cavity 116. Crimp 162 and wire 160 may advance through lumen 120, while biasing element 108 may be too large in diameter to advance through lumen 120. Tab 149 may help block or otherwise prevent biasing element 108 from entering recess 119 when release tab 145 is in a deactuated position or configuration. When spool 111 is in the engaged configuration, an end effector 170 of medical device 100 may be actuated by pulling spool 111 proximally. Pulling spool 111 proximally may pull wire 160 proximally. As spool 111 is pulled proximally, an increasing resistance may be felt by the user up until a click sound and/or tactile or audible feedback is exhibited by medical device 100, for example, indicating to the user that end effector 170 has been actuated and/or deployed. Pulling wire 160 proximally may actuate the end effector. Sheath 139 may be fixed relative to distal tip 133 so that pulling wire 160 does not move sheath 139. In some aspects, end effector 170 may be a hemostasis clips or a biopsy forceps, and movement of spool 111, and thus wire 160, may open, close, or otherwise control or actuate end effector 170.

Release tab 145 may be actuated (e.g., to transition tab 145 to an actuated position or configuration) by applying a force to lever portion 146 of release tab 145 so that tab 149 is moved radially outward from axis A of medical device 100. This movement may allow crimp 162 and wire 160 to be advanced into recess 119. After crimp 162 is received within recess 119, release tab 145 may be deactuated (e.g., transitioned to the deactuated position or configuration) by releasing the force so that tab 149 returns to its original, deactuated position or configuration by the elasticity of the material of pivot bar 151, or in the alternative, the user may manually move release tab 145 to its original, deactuated position or configuration In another alternative, release tab 145 may include a biasing element (not shown) to move release tab 145 to its original, deactuated position or configuration. Tactile or auditory feedback may be sensed by the user when crimp 162 enters recess 119 or when crimp 162 contacts the distal facing surface at the proximal end of recess 119. As tab 149 returns to its original, deactuated position or configuration, a portion of wire 160 distal of crimp 162 is received within notch 150. When crimp 162 is proximal tab 149 and within recess 119, crimp 162 may be in a locked configuration. As spool 111 is manipulated (e.g., moved proximally and/or distally) during a procedure, crimp 162 may contact tab 149 and the distal facing surface at the proximal end of recess 119. Crimp 162 may be biased against tab 149 by biasing element 108.

Spool 111 may be disengaged or uncoupled from main body 109 and slot 124. For example, spool 111 may be disengaged or uncoupled from main body 109 and slot 124 by manually pulling apart first spool component 114 and second spool component 115 and/or by disengaging snap 121 (e.g., depressing protrusion 122b of cantilevered portion 122 radially inward relative to slot 123). Spool 111 may be disengaged or uncoupled from disposable portion 132. For example, spool 111 may be disengaged or uncoupled from disposable portion 132 by applying a force to lever portion 146 and then pulling spool 111 in the proximal direction relative to crimp 162 and/or slot 124, so that crimp 162 exits through recess 119, lumen 120, and cavity 116. Alternatively or additionally, first spool component 114 and second spool component 115 may be disengaged or uncoupled from one another, which may also disengage or uncouple spool 111 from main body 109 and slot 124 and disengage or uncouple spool 111 from disposable portion 132.

After spool 111, handle 103 and main body 109 has been used in a procedure, or after spool 111, handle 103, and main body 109 have been otherwise contaminated, spool 111, handle 103 and main body 109 may be configured to be disinfected and reused with another disposable portion 132. Further, disposable portion 132 may be discarded after use and/or contamination and a new disposable portion 132 may be provided for a future procedure.

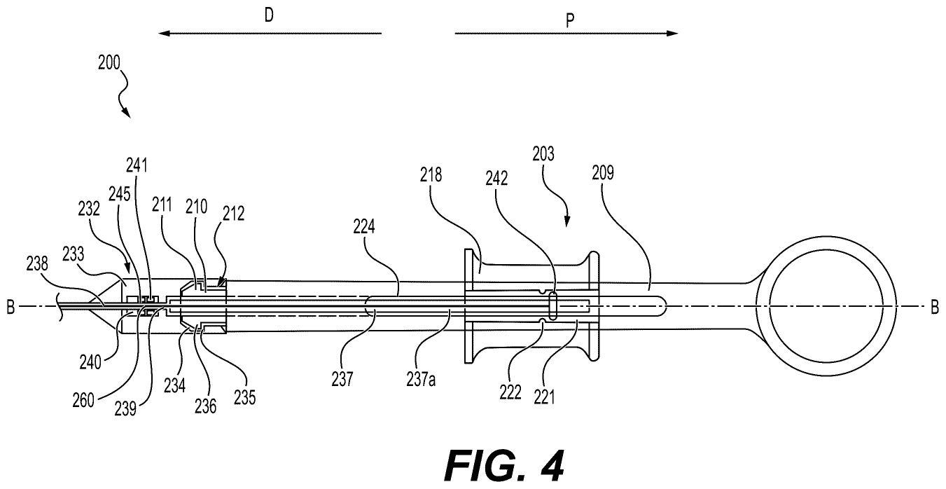

FIG. 4 depicts a portion of a medical device 200. Medical device 200 may have any of the features of medical device 100, or any of the other medical devices disclosed within this disclosure. Medical device 200 may include a handle 203 having a main body 209 and a slot 224. Handle 203 may include a spool 218 releasably engageable with main body 209 and slot 224. Handle 203 and spool 218 may have any of the features of handle 103 and spool 111, respectively. Medical device 200 may include a disposable portion 232 Disposable portion 232 may include a sheath 237 with a lumen configured to receive a wire 260. A couplable portion 237a of sheath 237 may be positioned within slot 224. Sheath 237 may be stationary relative to main body 209, while wire 260 may be moved relative to main body 209/sheath 237 via spool 218. Disposable portion 232 may have any of the features of disposable portion 132. Disposable portion 232 may include a distal tip 233. Distal tip 233 may include a sheath 245 with a lumen configured to receive wire 260. Wire 260 may be moveable relative to sheath 245. A proximal portion of sheath 245 may be anchored within distal tip 233. A distal portion of sheath 245 may extend to an end effector (such as end effector 170).

Main body 209 may include a protrusion 210 that protrudes distally from a distal end of main body 209. Distal tip 233 may include a cavity 234 configured to receive protrusion 210. Protrusion 210 may include a tab 211 that extends radially outward relative to a central longitudinal axis (labeled as axis B in FIG. 4) of medical device 200. Cavity 234 may include a recess 235 configured to tab 211. Cavity 234 may include a tab or shelf 236 that extends radially inward relative to axis B of medical device 200. Protrusion 210 may include a recess 212 configured to receive tab 236. Distal tip 233 may be coupled to main body 209 by inserting protrusion 210 into cavity 234. When distal tip 233 is coupled to main body 209, a proximal facing surface of tab 211 may contact a distal facing surface of tab 236 forming a snap fit configuration. Distal tip 233 may be decoupled from main body 209 by manually pulling apart distal tip 233 and main body 209. In some embodiments, protrusion 210 may form an interference fit with cavity 234. It should be understood that in alternate embodiments, distal tip 233 may include protrusion 210 and main body 209 may include cavity 234.

Distal tip 233 may include a lumen 238 along axis B. Lumen 238 may be configured to receive a portion of wire 260. Distal tip 233 may include a channel 239 adjacent and distal to cavity 234. Distal tip 233 may include a chamber 240. Chamber 240 may be positioned distally relative to channel 239 and cavity 234. Lumen 238, channel 239, cavity 234, and chamber 240 may all be in communication with one another. Couplable portion 237a may be received by slot 224 and spool 218. Sheath 237 may contact a proximal facing surface at a distal end of channel 239. Chamber 240 may include a crimp 241. Crimp 241 may have any of the features of crimp 141. Crimp 241 may be attached or fixed to a portion of sheath 245 to prevent movement of sheath 245 relative to distal tip 233.

Spool 218 may have any of the features of spool 111. Although not shown, spool 218 may include a first spool component and a second spool component, as discussed above. Spool 218 may include a channel 221. Channel 221 may extend along axis B of spool 218. Channel 221 may be configured to receive a portion of couplable portion 237a of sheath 237 and/or wire 260. Channel 221 may include a radially inwardly protruding flange 222, for example, extending radially inward along an inner circular surface of spool 218. Alternatively, the flange 222 may be replaced with one or more radially inwardly protruding tabs. Sheath 237 may include a flange 242. Flange 242 may extend radially outward relative to axis B. Wire 260 may be partially embedded or fixed to sheath 237, so that moving sheath 237 moves wire 260. Flange 242 may be fixed relative to sheath 237, and wire 260 may be configured to move with sheath 237, so that moving sheath 237 proximally via spool 218 also moves wire 260 proximally. Flange 242 and flange 222 may contact one another during actuation of spool 218. Flange 222 of channel 221 may be positioned distally of flange 242 of sheath 237, so that pulling spool 218 in a proximal direction causes flange 242, sheath 237, and wire 260 to be pulled in the proximal direction via contacting flange 222 pushing flange 242 in the proximal direction. Additionally, although not shown, in some aspects, spool 218 may include two flanges 222, for example, spaced apart longitudinally, and flange 242 may be positioned longitudinally between the two flanges 222 to operatively couple spool 218 with a portion of sheath 237 and/or wire 260.

In an alternate embodiment, flange 242 may be moveable relative to sheath 237 (e.g., couplable portion 237a). Wire 260 may be couplable to flange 242, so that when flange 242 is coupled to wire 260 and flange 242 is moved proximally by spool 218, wire 260 is moved proximally relative to sheath 237 and/or main body 209. Flange 242 and flange 222 may contact one another during the actuation of spool 218. Flange 222 of channel 221 may be positioned distally of flange 242 of sheath 237 so that pulling spool 218 in a proximal direction would cause flange 242 to be pulled in the proximal direction via contacting flange 222 pushing flange 242 in the proximal direction.

Spool 218 may be formed from a first spool component and a second spool component, for example, like spool 111. First spool component and second spool component may be transitioned to an engaged configuration about slot 224 in similar manner to components 114, 115 of spool 111. First spool component and second spool component may be transitioned to the engaged configuration by aligning components about main body 209 so that a portion of each component is positioned within slot 224 and at least one portion of each component is positioned outside slot 224 and around main body 209. First spool component and second spool component may be positioned along slot 224 so that flange 222 positioned distally to flange 242. The spool components may be disengaged from one another and from main body 209 and slot 224 by manually pulling apart the first and second spool components. After disengaging spool 218, sheath 237 and wire 260 may be removed via slot 224.

When spool 218 is in the engaged configuration, an end effector (for example, end effector 170) of medical device 200 may be actuated or otherwise controlled by pulling spool 218 proximally or otherwise manipulating spool 218. Pulling spool 218 proximally may pull flange 242, and by extension, wire 260, proximally. Pulling wire 260 proximally may actuate the end effector (for example, end effector 170). In some aspects, end effector 170 may be a hemostasis clips or a biopsy forceps, and movement of spool 218, and thus wire 260, may open, close, or otherwise control or actuate end effector 170. Although not shown, medical device 200 may include a biasing element, such as a spring, configured to urge spool 218 toward a neutral and/or distal position. The biasing element may also help urge flange 242 and sheath 237 to its original position prior to being pulled proximally via spool 218. The biasing element may be positioned proximally of spool 218 and/or flange 242. After being used in a procedure, disposable portion 232 may be discarded and replaced with another disposable portion 232. Spool 218 and main body 209 may be configured to be cleaned and/or be reusable in future procedures. In an alternate embodiment, flange 242 may be positioned distally to flange 222 so that urging spool 218 distally would urge flange 242 distally and thereby urge wire 260 distally to either open or close an end effector positioned at a distal end of wire 260. In this alternate embodiment, a biasing element may be positioned distal to flange 242 to urge flange 242 in a proximal direction after deactuating spool 218.

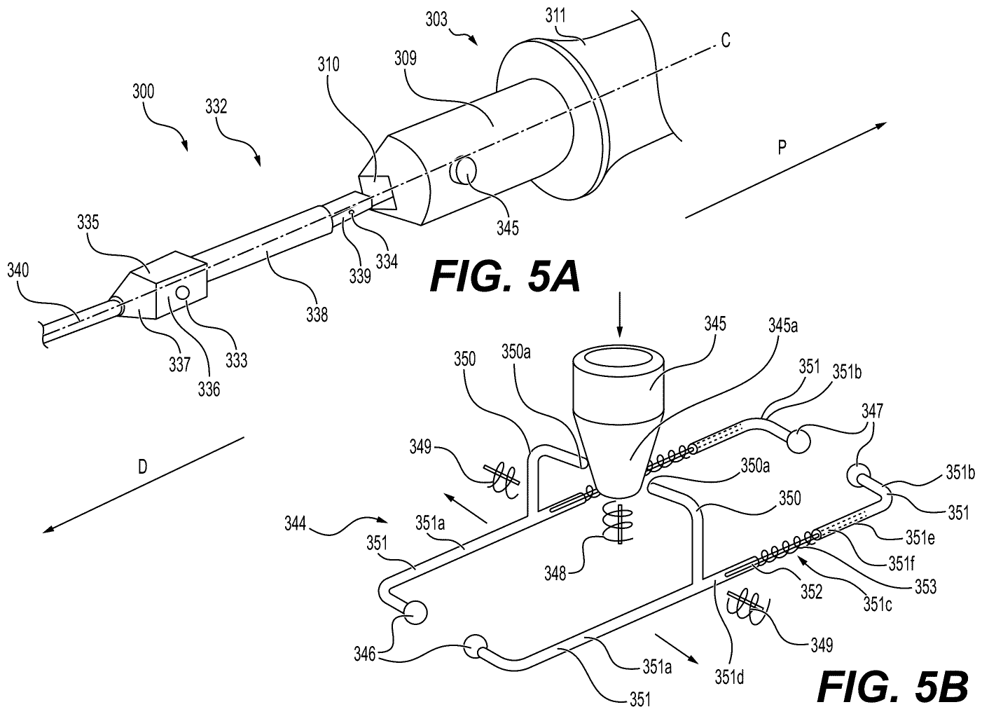

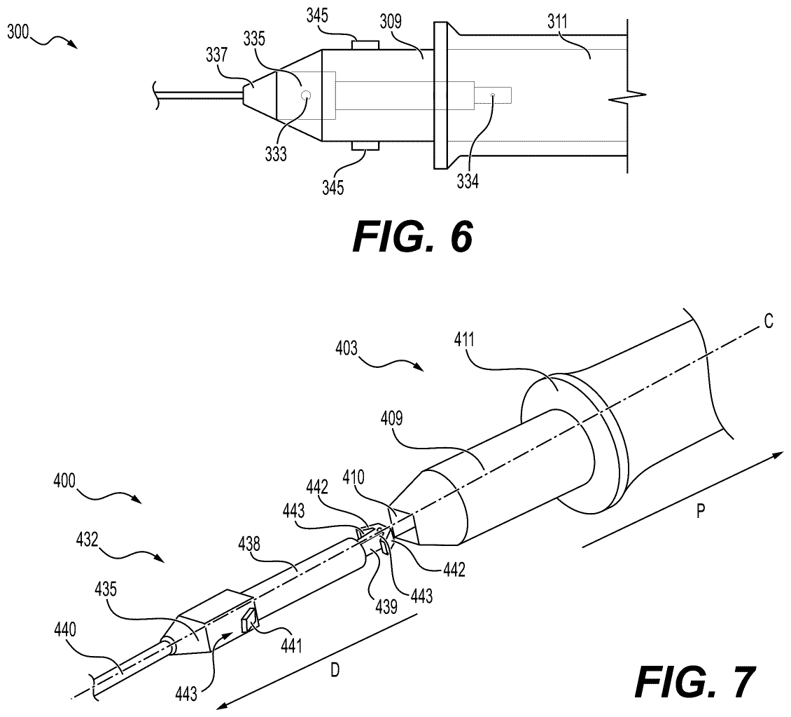

FIGS. 5A, 5B, and 6 depict various aspects of a medical device 300. Medical device 300 may have any of the features of medical devices 100, 200. Medical device 300 may include a main body 309. Main body 309 may include a slot (not shown). Medical device 300 may include a spool 311 releasably engageable with main body 309 and the slot. Medical device 300 may include a disposable portion 332. Disposable portion 332 may include an actuation wire (such as wire 160) for actuating an end effector (such as end effector 170). Pulling spool 311 proximally may pull wire proximally, actuating the end effector. Spool 311 have any of the features of spool 218 and spool 111.

Medical device 300 may include a locking mechanism 344 (FIG. 5B). A portion (a distal portion) of locking mechanism 344 may be positioned within main body 309, and another portion of locking mechanism 344 may be positioned within spool 311 and/or may be moveable with spool 311 along axis C. Locking mechanism 344 may be a ball lock. Locking mechanism 344 may include an actuator 345, such as a button. Button 345 may actuate one or more protrusions, such as a first protrusion 346 and a second protrusion 347. First and second protrusions 346, 347 may be spherical in shape, but are not limited to being spherical in shape and may be any shape. In some aspects, locking mechanism 344 may include two first protrusions 346 and two second protrusions 347. Locking mechanism 344 may include one or more lateral arms 350 and one or more longitudinal arms 351. One or more lateral arms 350 may include a first end 350a and a second end.

One or more lateral arms 350 may include a bend between first end 350a and the second end, angling the second end toward a central longitudinal axis (labeled as axis C in FIG. 5A and labeled as axis D in FIG. 7) of medical device 300. One or more longitudinal arms 351 may include one or more first ends and a second end. The second end of one or more longitudinal arms 351 may be connected to the second end of the one or more lateral arms 350. First and second protrusions 346, 347 may be connected to the one or more first ends of the one or more longitudinal arms 351. One or more longitudinal arms 351 may include a bend near first and second protrusions 346, 347, angling the first and second protrusions toward 346, 347 toward axis C.

One or more longitudinal arms 351 may include one or more distal longitudinal arms 351a and one or more proximal longitudinal arms 351b. One or more distal longitudinal arms 351a may be positioned in main body 309 and may be positioned near a distal end of main body 309. One or more distal longitudinal arms 351a may not be moveable along axis C. At least a portion of one or more proximal longitudinal arms 351b may be positioned within, moveable with and/or coupled to spool 311. One or more proximal longitudinal arms 351b may include a gap 351c along a longitudinal length of each of the one or more proximal longitudinal arms 351b. One or more proximal longitudinal arms 351b may include a rod 352. Rod 352 may be embedded or fixed to a distal portion 351d of each of one or more proximal longitudinal arms 351b, the distal portion 351d being distal to gap 351c. A proximal portion 351e of each of one or more proximal longitudinal arms 351b may include a hole or cavity 351f configured to receive at least a portion of rod 352. Proximal portion 351e may be configured to slide along a longitudinal axis of rod 352 so that moving spool 311 proximally moves proximal portion 351e proximally and the actuation wire proximally, and so that moving spool 311 distally moves proximal portion 351e distally and the actuation wire distally. Locking mechanism 344 may include a biasing element 353 extending along rod 352. Biasing element 353 is configured to urge proximal portion 351e in a proximal direction. When biasing element 353 urges proximal portion 351e in proximal direction, biasing element 353 may also urge spool 311 and the actuation wire proximally.

Depressing button 345 (as shown by the downward arrow in FIG. 5B) may cause a tapered portion 345a of button 345 to contact first end 350a of the one or more lateral arms 350. Additionally, depressing button 345 may cause one or more lateral arms 350 and longitudinal arms 351 to move radially outward relative to axis C (as shown by the radially outward arrows in FIG. 5B). As tapered portion 345a is depressed, the first end 350a of one or more lateral arms 350 contact tapered portion 345a and are gradually pushed outward as a diameter of tapered portion 345a increases relative to the first end of one or more lateral arms. Depressing button 345 may cause first protrusion 346 and second protrusion 347 to move radially outward relative axis C of medical device 300. Releasing button 345 may cause first protrusion(s) 346 and second protrusion(s) 347 to move radially inward, returning to an original position. Button 345 may be biased via a spring 348. Button 345 may be biased radially outward via spring 348 relative to axis C. One or more lateral arms 350, and by extension first protrusion 346 and second protrusion 347 and one or more longitudinal arms 350, may be biased radially inward via one or more springs 349. Disposable portion 332 may include one or more recesses, such as first recess 333 and second recess 334. First recess 333 may be sized and shaped to receive first protrusion 346 and second recess 334 may be configured to receive second protrusion 347. Engaging protrusions 346, 347 with recesses 333, 334 may couple disposable portion 332 to handle 303/main body 309 and may couple disposable portion 332 to spool 311.

Disposable portion 332 may include a hub 335. Hub 335 may include a rectangular portion 336 and a tapered portion 337 extending distally from rectangular portion 336. Disposable portion 332 may include a hollow, tubular member 338 extending proximally from a proximal end of hub 335. Disposable portion 332 may include a proximal tip 339. Proximal tip 339 may at least partially extend through tubular member 338. Alternatively, proximal tip 339 may extend through tubular member 338 and at least partially extend into hub 335 or extend to hub 335. Proximal tip 339 may be moveable relative to tubular member 338 and/or hub 335 along axis C.

First recess 333 may be positioned on hub 335 and second recess 334 may be positioned on proximal tip 339. Disposable portion 332 may include a sheath 340 extending distally from hub 335. A proximal portion of sheath 340 may be embedded within hub 335. Disposable portion 332 may define a lumen (not shown) extending along a central longitudinal axis of disposable portion 332 through one or more of sheath 340, hub 335, tubular member 338, and proximal tip 339. The lumen may be configured to receive an actuation wire. The actuation wire may extend through the lumen. The actuation wire may be moveable relative to tubular member 338, hub 335, and sheath 340 and may be moveable within the lumen. Sheath 340 may extend to a distal end of the actuation wire. Movement of the actuation wire distally or proximally may open or close an end effector positioned at a distal end of the actuation wire. The actuation wire may be coupleable or fixed to proximal tip 339 so that moving proximal tip 339 urges the actuation wire in the same direction. For example, when second protrusion 347 is received within second recess 334, actuating spool 311 distally may urge proximal tip 339 and the actuation wire distally. After deactuating spool 311, biasing element 353 may urge proximal tip 339 and the actuation wire proximally.

Main body 309 include a cavity 310. Cavity 310 may extend along a central longitudinal axis of main body 309 proximally from a distal end of main body 309. First protrusion 346 and second protrusion 347 may protrude within cavity 310. Cavity 310 may be sized and shaped to receive at least a portion of disposable portion 332. For example, cavity 310 may be sized and shaped to receive proximal tip 339, tubular member 338, and at least a portion of hub 335, such as rectangular portion 336. The distal end of main body 309 may taper distally to a distal opening of cavity 310. Tapered portion 337 may extend distally beyond the distal opening of cavity so that tapered portion 337 and the distal end of main body 309 form a continuous tapered surface.

Disposable portion 332 may be releasably engaged within main body 309 and spool 311 by applying a force to button 345 (e.g., radially inward), inserting disposable portion 332 into cavity 310, aligning first recess 333 with first protrusion 346 and aligning second recess 334 with second protrusion 347, and releasing the force applied to button 345 so that protrusions 346, 347 engage with the recesses 333, 334. In the alternative, disposable portion 332 may be engaged with main body 309 and spool 311 by applying sufficient force to disposable portion 332 to overcome the force of biasing elements 349, 348. Disposable portion 332 and/or main body 309 may be configured to provide auditory and/or tactile feedback to the user when the recesses and protrusions are aligned and/or when disposable portion 332 and main body 309 are engaged. Disposable portion 332 may be disengaged from main body 309 and spool 311 by applying a force to button 345 and removing disposable portion 332 from cavity 310. Main body 309 and spool 311 may then be sanitized and reused with a new disposable portion 332. FIG. 6 depicts a side view of medical device 300 with disposable portion 332 engaged with cavity 310. FIG. 6 shows two buttons 345, which may be used to actuate locking mechanism 344. It should be understood that locking mechanism 344 may include an additional two lateral arms 350 that extend in an opposite direction to the first two lateral arms 350 that may be actuated by the second button 345. It should be further understood that locking mechanism 344 may also include an additional biasing element 348 biasing the second button 345 radially outward relative to axis C. In some embodiments, medical device 300 may include a second locking mechanism 344 corresponding to second button 345 and make include second recesses 333, 334.

FIG. 7 depicts an alternate embodiment of medical device 300, a medical device 400. Medical device 400 may have any of the features of medical devices 300, 200, 100. Medical device 400 may include a locking mechanism similar to locking mechanism 344 where protrusions 346, 347 may be angled tabs extending proximally and radially inward. Instead of recess 333, hub 435 may include an angled tab 441 extending distally and radially outward. Similarly, instead of recess 334, proximal tip 439 may include an angled tab 442 extending distally and radially outward. Protrusion 346 may include a surface configured to engage with a surface of angled tab 441. Protrusion 347 may include a surface configured to engage with a surface of angled tab 442. Angled tabs 441, 442 may each include a recess 443 for receiving protrusions 346, 347, respectively. Disposable portion 432 may be prevented from exiting cavity 410 when protrusions 346, 347 are received within recesses 443. Instead of moving radially outward when actuator 345 is actuated, protrusions 346, 347 may move distally and/or radially outward. Similarly, instead of moving radially inward when actuator 345 is no longer being actuated, protrusions 346, 347 may move proximally and/or radially inward.

Disposable portion 432 may define a lumen (not shown) extending along a central longitudinal axis (axis D) of disposable portion 432 through one or more of sheath 440, hub 435, tubular member 438, and proximal tip 439. Proximal tip 439 may be moveable relative to tubular member 438 along axis D. One or more proximal longitudinal arms 351b may be positioned within and/or coupled to spool 411, so that when protrusion 347 and angled tab 442 are engaged, proximal tip 439 moves with spool 411.

The lumen of disposable portion 432 may be configured to receive an actuation wire. The actuation wire may extend through the lumen. The actuation wire may be moveable relative to tubular member 338, hub 335, and sheath 340 and may be moveable within the lumen. Sheath 440 may extend to a distal end of the actuation wire. Movement of the actuation wire distally or proximally may open or close an end effector positioned at a distal end of the actuation wire. The actuation wire may be coupleable or fixed to proximal tip 439 so that moving proximal tip 439 moves the actuation wire. For example, when second protrusion 347 is engaged with angled tab 442, actuating spool 411 proximally may urge both proximal tip 439 and the actuation wire proximally. After deactuating spool 411, biasing element 353 may urge both proximal tip 439 and the actuation wire distally. Alternatively, actuating spool 411 distally may urge both proximal tip 439 and the actuation wire distally. After deactuating spool 411, biasing element 353 may then urge both proximal tip 439 and the actuation wire proximally.

Disposable portion 432 may be releasably engaged with main body 409 and spool 411 by actuating actuator 345, inserting disposable portion 432 into cavity 410, aligning angled tabs 441, 442 within cavity 410 and spool 411, respectively, so that protrusions 346, 347 are received within recesses 443 when actuator 345 is no longer being actuated (in other words, so that protrusions 346, 347 are aligned with recesses 443). After aligning, disposable portion 432 may be locked in by deactuating actuator 345 so that protrusions 346, 347 move proximally and/or radially inward into recesses 443, thereby locking/releasably engaging disposable portion 432 within cavity 410. Disposable portion 432 may be disengaged with main body 409 and spool 411 by actuating actuator 345 so that protrusions 346, 347 move distally and/or radially outward, removing disposable portion 432 from cavity 410, and helping to stop the actuation of actuator 345. Any feature of the medical device 400 may be included in the primary embodiment of medical device 300.

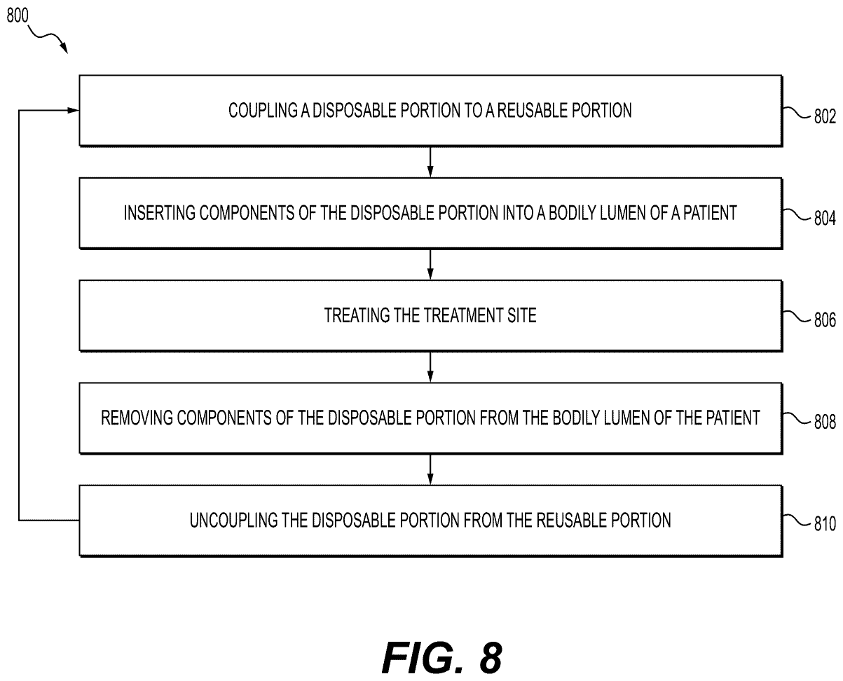

As seen in FIG. 8, this disclosure includes a method 800 for releasably engaging components of medical devices 100, 200, 300, and 400. Method 800 may include an initial step that includes coupling a moveable body (e.g., spools 111, 218, 311, 411) to a handle (e.g. main body 109, 209, 309, 409). For example, engaging first spool component 114 and second spool component 115 along slot 124 of main body 109. First spool component 114 and second spool component 115 may be secured to main body 109 via snap 121. Method 800 may include an initial step of removing the disposable portion (e.g., disposable portion 132, 232, 332, 432) from a sterile packaging. Method 800 may include an initial step that include assembling disposable portion. For example, method 800 may include assembling disposable portion 132 (e.g., sheath 139, wire 160, distal tip 133, and end effector 170).

Method 800 includes a step 802 of coupling a disposable portion to the reusable portion. Step 802 may include coupling the disposable portion to the moveable body of the handle of the reusable portion. For example, step 802 may include inserting extension 142 of distal tip 133 within chamber 129 of main body 109 and inserting sheath 139 and wire 160 through narrower lumen 127, narrow lumen 126, and wide portion 125 (e.g., through a lumen of main body 109 into slot 124). Alternatively, step 802 may include actuating release tab 145 and then inserting crimp 162 and wire 160 (e.g., proximal end 161a) through cavity 116, lumen 120 and then into recess 119. In this aspect, step 802 may include deactuating release tab 145, so that crimp 162 and proximal end 161a is contained within recess 119 and so that a portion of wire 160 is received within notch 150 of release tab 145. In another example, disposable portion 332 may be coupled with handle 303 by actuating locking mechanism 344 and inserting disposable portion 332 within cavity 310 and then deactuating locking mechanism 344.

Method 800 includes a step 804 that includes inserting components (e.g., wire, end effector, and sheath) of the disposable portion into a bodily lumen of a patient. For example, end effector 170 may be inserted into a port of an endoscope (or other scopes used in the medical field) and then introduced into a bodily lumen or manufactured cavity/orifice of a patient. In these aspects, at least a distal end of the disposable portion may be delivered to a treatment site.

Method 800 further includes a step 806 that includes treating the treatment site. For example, the moveable body (e.g., spool 111, 218, 311, 411) may be moved in a distal direction. Actuating (e.g., urging the spool 111, 218, 311, 411 in a distal direction) the moveable body may open or close an end effector of the medical device. Deactuating the moveable body may allow a biasing element to urge the moveable body back to an original, deactuated (e.g., pre-actuated) position and may open or close the end effector.

Method 800 includes a step 808 of removing the components of the disposable portion from the bodily lumen or manufactured cavity/orifice of the patient. For example, end effector may be retracted proximally toward a shaft of an endoscope. In some examples, the end effector may be used to capture tissue or other material from the treatment site, and removing the disposable portion may also help to remove the tissue or other material from the patient.

Method 800 includes a step 810 that includes uncoupling the disposable portion from reusable portion. For example, step 810 may further include actuating release tab 145 and then removing crimp 162 and wire 160 from spool 111 by urging spool 111 proximally. After wire 160 and crimp 162 are removed from spool 111, disposable portion may be manually removed from the handle by removing distal tip 133 from chamber 129. In another example, disposable portion 332 may be decoupled with handle 303 by actuating locking mechanism 344 and removing disposable portion 332 from cavity 310 and then deactuating locking mechanism 344. After use, disposable portions 132, 232, 332, 432 may be disposed. Additionally, in some aspects, handles 103, 203, 303, 403 may be cleaned and/or reused in later procedures with new disposable portions. Step 810 may also including disposing of the disposable portion

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a disposable portion, including a sheath and a wire disposed within a lumen of the sheath,
a handle, the handle comprising
a main body with a slot,
a releasably engageable moveable body, wherein the moveable body is formed by a first component and a second component, and wherein the first component and the second component are releasably engageable with both the slot and a proximal portion of the disposable portion,
wherein movement of the moveable body is configured to move the wire.

2. The medical device of claim 1, wherein the disposable portion includes an end effector, wherein the moveable body is configured to receive a proximal portion of the wire and movement of the moveable body is configured to actuate the end effector.

3. The medical device of claim 2, wherein the disposable portion includes a first crimp positioned at a proximal end of the wire, wherein the moveable body is configured to receive the first crimp.

4. The medical device of claim 3, wherein the first component includes a release tab.

5. The medical device of claim 4, wherein when the release tab is in an actuated configuration, the first crimp and proximal end of the wire are receivable within a recess of the second component.

6. The medical device of claim 5, wherein the release tab includes a tab, a slot defined through the tab, the slot sized and shaped to receive the wire and reject the first crimp.

7. The medical device of claim 6, wherein, when the first crimp is received within the recess and the release tab is in a deactuated configuration, the first crimp is prevented from moving in a distal direction by the tab.

8. The medical device of claim 6, wherein when the first crimp is received within the recess and the release tab is in a deactuated configuration, a portion of the wire is disposed within the slot of the tab.

9. The medical device of claim 3, wherein the disposable portion includes a removable distal tip, wherein the distal tip includes a lumen and a chamber, wherein the lumen and chamber are in communication with each other and are positioned along a central longitudinal axis of medical device, and wherein at least a portion of the wire is positioned within the lumen and the chamber.

10. The medical device of claim 9, wherein the disposable portion includes a second crimp positioned distally of the first crimp on the wire, the second crimp fixedly attached to a portion of the sheath and positioned within the chamber, wherein a dimension of the second crimp prevents the second crimp from exiting the chamber.

11. The medical device of claim 10, wherein the second crimp prevents movement of the sheath relative to the distal tip.

12. The medical device of claim 3, wherein a distal tip of the disposable portion includes a protrusion extending in a proximal direction from a proximal end of the distal tip, wherein the main body includes a chamber configured to receive the protrusion, wherein the distal tip is releasably attached to the main body when the protrusion is received within the chamber of the main body.

13. The medical device of claim 12, wherein the protrusion includes a flange and the chamber includes a flange, wherein the protrusion of the distal tip is received within the chamber of the main body, and wherein the flange of the protrusion and the flange of the chamber contact one another to prevent the protrusion from exiting the chamber.

14. The medical device of claim 13, wherein the flange of the protrusion extends radially outward relative to a central longitudinal axis of the medical device and the flange of the chamber extends radially inward relative to the central longitudinal axis of the medical device.

15. The medical device of claim 1, wherein the handle includes a locking mechanism configured to couple the disposable portion with the handle.

16. A medical device, comprising:
a disposable portion, the disposable portion including a distal tip, the distal tip including a cavity at a proximal end of the distal tip,
a handle, the handle comprising:
a main body with a slot, the main body including a protrusion extending distally from a distal end of the main body, the cavity configured to receive and contain the protrusion of the distal tip when the disposable portion is in an engaged configuration with the main body,
a moveable body,
wherein movement of the moveable body is configured to control an end effector of the medical device.

17. The medical device of claim 16, wherein the protrusion includes a tab extending radially outward relative to a central longitudinal axis of the medical device, wherein the cavity includes a tab extending radially inward relative to the central longitudinal axis of the medical device.

18. The medical device of claim 16, wherein the disposable portion includes a sheath, the sheath receivable within a channel of the moveable body, the sheath including a flange and the channel including a flange, the flange of the moveable body positioned distally to the flange of the sheath when sheath is received within the moveable body.

19. The medical device of claim 18, wherein the moveable body is formed from a first moveable body component and a second moveable body component, wherein the moveable body is releasably engageable to the main body and the slot.

20. A method of releasably engaging components of medical device, wherein the medical device includes a reusable portion including a handle with an actuator, wherein the actuator is a moveable body, wherein the medical device includes a disposable portion, the disposable portion including a wire and a crimp positioned at a proximal end of the wire, wherein the moveable body includes a release tab and a recess configured to receive the crimp, wherein a tab of the release tab is distal to the recess and blocks insertion of the crimp into the recess when the release tab is in an deactuated state, wherein in an actuated state the tab of the release tab no longer blocks insertion of the crimp into the recess, the method comprising:
coupling the disposable portion to the reusable portion;
inserting components of the disposable portion into a bodily lumen of a patient;
treating a treatment site;
removing components of the disposable portion from the bodily lumen of the patient; and
uncoupling the disposable portion from the reusable portion.
